(19) United States
(12) Patent Application Publication
(10) Pub. No.: US 2014/0249610 A1
(43) Pub. Date: Sep. 4, 2014

(54) REFRIGERANT CARTRIDGES FOR CRYOTHERAPEUTIC SYSTEMS AND ASSOCIATED METHODS OF MAKING AND USING

(71) Applicant: MEDTRONIC ARDIAN LUXEMBOURG S.A.R.L., Luxembourg (LU)

(72) Inventor: Eric Ryba, Santa Rosa, CA (US)

(21) Appl. No.: 14/346,695

(22) PCT Filed: Oct. 5, 2012
(86) PCT No.: PCT/US12/59042

§ 371 Date: Mar. 21, 2014

Related U.S. Application Data
(60) Provisional application No. 61/545,052, filed on Oct. 7, 2011.

(51) Int. Cl. A61F 7/12; B65B 3/04

(52) U.S. Cl. CPC A61F 7/12; B65B 3/04; A61F 2007/126
USPC 607/104; 53/425

(57) ABSTRACT

Refrigerant cartridges for cryotherapeutic neuromodulation and associated systems and methods are disclosed herein. In one embodiment, for example, a cryotherapeutic system includes a shaft having a proximal portion, a distal portion and a supply lumen along at least a portion of the shaft. The shaft can be configured to locate the distal portion intravascularly at a treatment site. The supply lumen can be configured to receive a refrigerant from a refrigerant cartridge. The refrigerant in the refrigerant cartridge can have a moisture concentration of at most 10 ppm. The system can further include a cooling assembly at the distal portion of the shaft that has an expansion chamber in fluid communication with the supply lumen.

### CROSS-REFERENCE TO RELATED APPLICATION(S)

This application claims the benefit of and priority to pending U.S. Provisional Patent Application No. 61/545.052, filed Oct. 7, 2011, which is incorporated by reference herein in its entirety.

### TECHNICAL FIELD

The present disclosure relates to cryotherapeutic neuromodulation and, more particularly, to refrigerant cartridges for cryotherapeutic neuromodulation and associated methods and systems.

### BACKGROUND

Hypertension, heart failure, chronic kidney disease, insulin resistance, diabetes and metabolic syndrome represent significant and growing health issues. Current therapies for such conditions include pharmacological, non-pharmacological and device-based approaches. Despite this variety of treatment options, high blood pressure and associated diseases remain uncontrolled for a large population.

Recently, the reduction of sympathetic renal nerve activity has been shown to reduce blood pressure in patients with treatment-resistant hypertension. Radiofrequency ablation or cryogenic cooling may be used to initiate therapeutically-effective renal neuromodulation via partial or full denervation. During cryogenic cooling, for example, a refrigerant may be circulated through a catheter to achieve cryogenic temperatures at a distal tip of the catheter that can be used to modulate neural fibers that innervate a kidney. The refrigerant used in such cryo-catheters is typically a compressed or condensed gas (e.g., nitrous oxide, carbon dioxide, etc.). In many cardiac applications, the compressed or condensed refrigerant gas is stored in a liquid state within a large reservoir (e.g., liter-sized cylinders) to accommodate complex refrigeration cycles. The large reservoirs generally contain enough liquid refrigerant to perform several procedures.

Smaller refrigerant cartridges of highly compressed or condensed gas have been used in non-medical applications, such as food processing (e.g., for the preparation of whipped cream), safety (e.g., to quickly inflate life vests), power (e.g., to drive mechanical actuators using argon or nitrogen pressurized gas), and recreation (e.g., in paintball and BB guns, etc.). Small gas refrigerant cartridges typically include relatively large amounts of water vapor and other contaminants (e.g., exceeding 60 parts per million (ppm)), which can cause them to be unsuitable for use in medical procedures.

### BRIEF DESCRIPTION OF THE DRAWINGS

FIG. 1 is a partially schematic view of a cryotherapeutic system configured in accordance with an embodiment of the present technology.

FIG. 2 is a partially schematic view of the cryotherapeutic system of FIG. 1 during a stage of cryotherapeutic renal neuromodulation in accordance with an embodiment of the present technology.

FIG. 3 is a block diagram illustrating a method of preparing a refrigerant cartridge in accordance with an embodiment of the present technology.

FIG. 4 is a conceptual diagram illustrating the sympathetic nervous system and how the brain communicates with the body via the sympathetic nervous system.

FIG. 5 is an enlarged anatomical view illustrating nerves innervating a left kidney to form a renal plexus surrounding a left renal artery.

FIGS. 6 and 7 are anatomical and conceptual views, respectively, illustrating a human body including a brain and kidneys and neural efferent and afferent communication between the brain and kidneys.

FIGS. 8 and 9 are anatomic views illustrating, respectively, an arterial vasculature and a venous vasculature of a human.

### DETAILED DESCRIPTION

The present technology is directed toward refrigerant cartridges for cryotherapeutic systems and associated methods. In several embodiments, a refrigerant cartridge may include a liquid refrigerant with a moisture concentration of not more than 10 ppm (e.g., 6 ppm). At the low temperatures used in cryotherapeutic renal neuromodulation (e.g., −60° C. and lower), excessive levels of moisture in the liquid refrigerant may reach the dew point, freeze, obstruct small supply lines, and eventually cause the cryotherapeutic system to fail. Accordingly, conventional refrigerant cartridges with refrigerants having moisture concentrations exceeding 60 ppm may be unsuitable for use with cryotherapeutic renal neuromodulation systems. Several embodiments of refrigerant cartridges disclosed herein are expected to reduce the likelihood of system failure (e.g., as a result of supply line blockages) during cryotherapeutic renal neuromodulation.

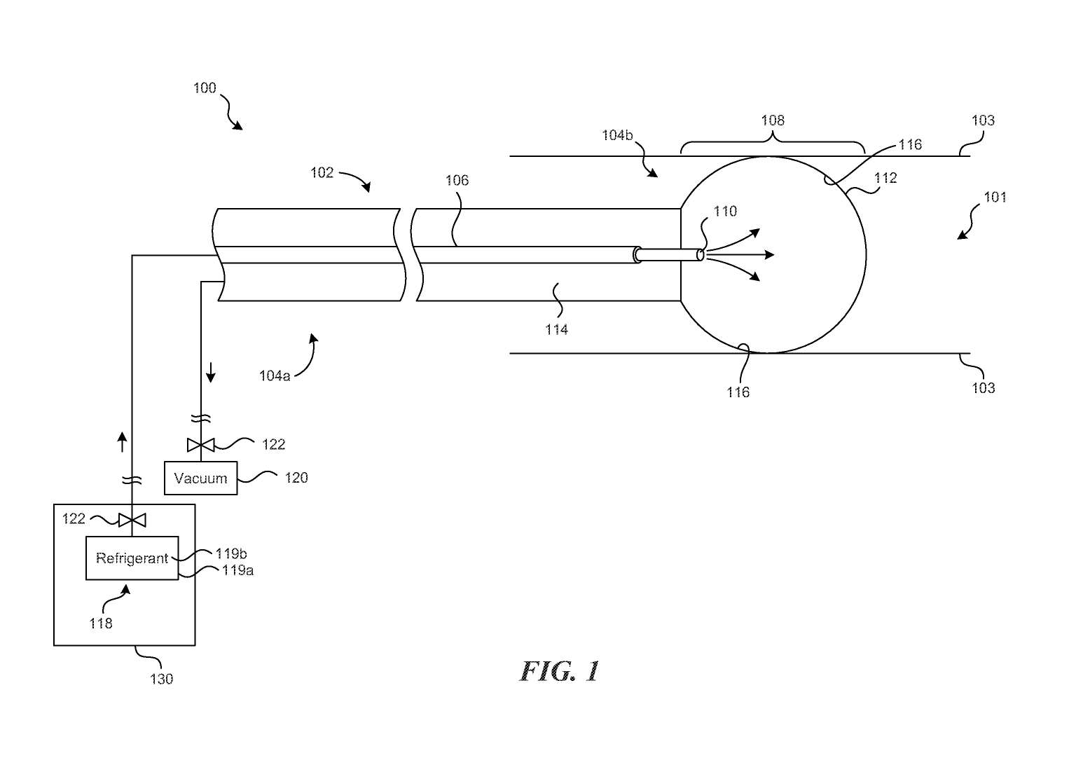

In the following description, certain specific details are set forth and in FIGS. 1-9 to provide a thorough understanding of various embodiments of the technology. For example, many of the embodiments are described below with respect to devices for cryotherapeutic renal neuromodulation via renal arteries. The present technology, however, may be used in other cryotherapeutic applications, such as cryogenically-induced nerve or tissue modulation in other small, peripheral vessels and/or other portions of the vasculature. Other details describing well-known structures and systems often associated with refrigeration, cryotherapy and associated devices have not been set forth in the following disclosure to avoid unnecessarily obscuring the description of the various embodiments of the technology. A person of ordinary skill in the art, therefore, will accordingly understand that the technology may have other embodiments with additional elements, or the technology may have other embodiments without several of the features shown and described below with reference to FIGS. 1-3.

The terms “distal” and “proximal” are used in the following description with respect to a position or direction relative to the operator or the operator's control device (e.g., a handle assembly). “Distal” or “distally” are a position distant from or in a direction away from the operator or the operator's control device. “Proximal” and “proximally” are a position near or in a direction toward the operator or the operator's control device.

### I. CRYOTHERAPY AND RENAL NEUROMODULATION

Cryotherapeutic systems and components of cryotherapeutic systems configured in accordance with embodiments of the present technology can be configured for renal neuromodulation, i.e., the partial or complete incapacitation or other effective disruption of nerves innervating the kidneys. In particular, renal neuromodulation can include inhibiting, reducing, and/or blocking neural communication along neural fibers (i.e., efferent and/or afferent nerve fibers) innervating the kidneys. Such incapacitation can be long-term (e.g., permanent or for periods of months, years, or decades) or short-term (e.g., for periods of minutes, hours, days, or weeks). Renal neuromodulation can contribute to the systemic reduction of sympathetic tone or drive. Accordingly, renal neuromodulation is expected to be useful in treating clinical conditions associated with systemic sympathetic overactivity or hyperactivity, particularly conditions associated with central sympathetic overstimulation. Renal neuromodulation is expected to efficaciously treat hypertension, heart failure, acute myocardial infarction, metabolic syndrome, insulin resistance, diabetes, left ventricular hypertrophy, chronic and end-stage renal disease, inappropriate fluid retention in heart failure, cardio-renal syndrome, polycystic kidney disease, polycystic ovary syndrome, osteoporosis, and sudden death, among others. Furthermore, renal neuromodulation can potentially benefit a variety of organs and bodily structures innervated by sympathetic nerves. A more detailed description of pertinent patient anatomy and physiology is provided below.

Various techniques can be used to partially or completely incapacitate neural pathways, such as those innervating the kidneys. Cryotherapy, for example, includes cooling tissue at a target site in a manner that modulates neural function. The mechanisms of cryotherapeutic tissue damage include, for example, direct cell injury (e.g., necrosis), vascular injury (e.g., starving the cell from nutrients by damaging supplying blood vessels), and sublethal hypothermia with subsequent apoptosis. Exposure to cryotherapeutic cooling can cause acute cell death (e.g., immediately after exposure) and/or delayed cell death (e.g., during tissue thawing and subsequent hyperperfusion). Several embodiments of the present technology include cooling a structure at or near an inner surface of a renal artery wall such that proximate (e.g., adjacent or nearby) tissue is effectively cooled to a depth where sympathetic renal nerves reside. For example, the cooling structure can be cooled to the extent that it causes therapeutically effective cryogenic renal neuromodulation. Sufficiently cooling at least a portion of a sympathetic renal nerve is expected to slow or potentially block conduction of neural signals to produce a prolonged or permanent reduction in renal sympathetic activity.

Cryotherapy has certain characteristics that can be beneficial for renal neuromodulation. For example, rapidly cooling tissue can provide an analgesic effect such that cryotherapies may be less painful than ablating tissue at high temperatures. Cryotherapies may thus require less analgesic medication to maintain patient comfort during a procedure compared to heat-ablation procedures. Additionally, reducing pain can reduce patient movement and thereby increase operator success or reduce procedural complications. Cryotherapy also typically does not cause significant collagen tightening, and therefore is not typically associated with vessel stenosis. Cryotherapies generally include cooling at temperatures that cause cryotherapeutic applicators to adhere to moist tissue. This can be beneficial because it can promote stable, consistent, and continued contact during treatment. The typical conditions of treatment can make this an attractive feature because, for example, a patient can move during treatment, a catheter associated with an applicator can move, and/or respiration can cause the kidneys to rise and fall and thereby move the renal arteries. In addition, blood flow is pulsatile and causes the renal arteries to pulse. Adhesion associated with cryotherapeutic cooling also can be advantageous when treating short renal arteries in which stable intravascular positioning can be more difficult to achieve.

### II. CRYOTHERAPEUTIC SYSTEMS WITH REFRIGERANT CARTRIDGES

FIG. 1 is a partially schematic view of a cryotherapeutic system 100 (“system 100”) configured in accordance with an embodiment of the present technology. The system 100 can include an elongated catheter body or shaft 102 having a proximal portion 104a and a distal portion 104b. A supply tube or lumen 106 can extend along at least a portion of the shaft 102 and be configured to transport a refrigerant in at least a partially liquid state to the distal portion 104b of the shaft 102. At the distal portion 104b, the system 100 includes a cooling assembly 108 that can have an expansion chamber 112 in fluid communication with the supply lumen 106 via an orifice 110. An exhaust tube or lumen 114 (e.g., defined by a portion of the shaft 102) can be placed in fluid communication with the expansion chamber 112 (e.g., a balloon, an inflatable body, etc.) such that it can return the refrigerant to the proximal portion 104a of the shaft 102. For example, in one embodiment, a vacuum 120 at the proximal portion 104a of the shaft 102 may be used to exhaust the refrigerant from the expansion chamber 112 via the exhaust lumen 114. In other embodiments, the refrigerant may be transported to the proximal portion 104a of the shaft 102 using other suitable mechanisms known to those having skill in the art.

During cryotherapy, the orifice 110 (e.g., defined by a capillary tube and/or other small opening) can restrict refrigerant flow to provide a high pressure differential between the supply lumen 106 and the expansion chamber 112, thereby facilitating the expansion of the refrigerant to the gas phase within the expansion chamber 112. In other embodiments, the orifice 110 can be an open end of the supply lumen 106 that has the same diameter as the supply lumen 106. The pressure drop as the liquid refrigerant passes through the orifice 110 causes the refrigerant to expand to a gas and reduces the temperature to a therapeutically effective temperature that can modulate neural fibers proximate a treatment site within a vessel 101. In the illustrated embodiment, for example, the expansion chamber 112 includes heat transfer portions 116 that contact and cool vessel walls 103 at a rate sufficient to cause cryotherapeutic renal neuromodulation. In various embodiments, the back pressure of the system 100 can be adjusted via the vacuum 120 or other suitable mechanism to regulate the pressure and temperature within the expansion chamber 112.

The refrigerant used for cryogenic cooling in the system 100 can be a compressed or condensed gas that is stored in at least a substantially liquid phase, such as nitrous oxide (N₂O), carbon dioxide (CO₂), hydrofluorocarbon (e.g., FREON made available by E. I. du Pont de Nemours and Company of Wilmington, Del.), and/or other suitable fluids that can be stored at a sufficiently high pressure to be in at least a substantially liquid phase at about ambient temperature. For example, R-410A, a zeotropic, but near-azeotropic mixture of difluoromethane (CH₂F₂; also known as HFC-32 or R-32) and pentafluoroethane (CHF₂CF₃; also known as HFC-125 or R-125), can be in at least a substantially liquid phase at about ambient temperature when contained at a pressure of about 1.45 MPa (210 psi). Under proper conditions, these refrigerants can reach cryotherapeutic temperatures at or near their respective normal boiling points (e.g., approximately −88° C. for nitrous oxide) to effectuate renal neuromodulation.

As shown in the FIG. 1, the shaft 102 and associated lumens are relatively small in outer diameter (e.g., 6 Fr or smaller) to access renal arteries (e.g., typically having an inner diameter of about 2 mm (0.079 inch) to 10 mm (0.394 inch)), and the supply lumen 106 and associated orifice 110 are be sized to generate sufficiently low cryogenic temperatures (e.g., about −60° C. or lower within the expansion chamber 112) to effectuate renal neuromodulation. For example, the orifice 110 may be defined by a distal end of a capillary tube having an opening of 0.102 mm (0.004 inch) to 0.203 mm (0.008 inch) (e.g., 0.127 mm (0.005 inch)). If the refrigerant supplied to such small lumens has a contaminant concentration above a threshold level, the lumens can become partially or fully obstructed. Typically, the primary contaminant is water vapor, which can condense out of the refrigerant stream as the liquid expands at the orifice 110 when the temperature drops below the dew point for the concentration of moisture in the refrigerant. The condensed refrigerant can freeze and obstruct the supply lumen 106 or orifice 110. Accordingly, when the system 100 uses cryogenic temperatures of approximately −60° C., the refrigerant can have a moisture concentration of equal to or less than approximately 10 ppm to prevent the moisture from condensing. When the system 100 operates at temperatures of less than −80° C., as may be the case when using a nitrous oxide refrigerant, the refrigerant can have a moisture concentration of less than approximately 6 ppm (e.g., 5 ppm, 1 ppm, 250 parts per billion, etc.) to inhibit condensation. In other embodiments, the system 100 can operate at higher temperatures (e.g., −40° C.) and/or the refrigerant can have a higher moisture concentration (e.g., between 10 ppm and 40 ppm).

The system 100 can therefore include a refrigerant cartridge 118 having a container 119a with a refrigerant 119b stored therein that has a relatively low moisture concentration to prevent or limit the likelihood of water vapor condensing out of the refrigerant during cryotherapeutic renal neuromodulation. Unlike cryogenic devices used to treat arthrosclerosis that operate at temperatures between −5° C. and −15° C., the system 100 of FIG. 1 operates at temperatures of −40° C., −60° C., −80° C., or lower. Accordingly, the refrigerant 119b in the container 119a can have a moisture concentration that is low enough to prevent or limit the likelihood of water vapor condensing at the operating temperatures of the system 100 (e.g., −40° C., −60° C., −80° C.). For example, the refrigerant 119b can have a moisture concentration less than 10 ppm (e.g., 6 ppm) for systems 100 operating at −60° C. In other embodiments, the system 100 may reach temperatures of less than −85° C., and the refrigerant 119b can have a moisture concentration of 5 ppm or less (e.g., 225 ppb). The duration of the treatment (e.g., multiple cooling applications) may further decrease the threshold moisture concentration of the refrigerant 119b.

Other contaminants that may condense or otherwise obstruct the system 100 during cryotherapy (e.g., higher weight hydrocarbons) can be regulated such that the refrigerant 119b has a substantially high purity level. For example, the refrigerant 119b may have a purity of at least about 95% (e.g., 98%, 99%, 99.9%). However, it should be noted that contaminants having low boiling points similar to that of the refrigerant 119b (e.g., hydrocarbons with lower molecular weights) may not be closely regulated, as these contaminants may have a less detrimental overall effect than other contaminants. Accordingly, the selected purity level of the refrigerant 119b can be dependent at least to some extent on the impurities present.

As further shown in FIG. 1, in some embodiments the system 100 may operate and circulate the refrigerant with relatively simple valving (e.g., via valves 122 coupled to the supply and exhaust lumens 106 and 114), rather than complex refrigeration cycles that may necessitate large reservoirs of refrigerant fluid. Accordingly, the container 119a of the refrigerant cartridge 118 can be a small container including sufficient refrigerant for at least one treatment, and can be disposed of or refilled after treatment. In one embodiment, for example, the container 119a has a length of at most 110 mm (4.33 inches), a diameter of at most 30 mm (1.18 inches), and stores a minimum volume of 30 cc (1.83 cubic inches). In other embodiments, the container 119a can have other suitable dimensions. The small container 119a provides greater flexibility to the system 100 by allowing the refrigerant cartridge 118 to be moved easily during procedures and even set on the patient during treatment.

The refrigerant 119b within the container 119a is a compressed or condensed refrigerant in a high density liquid phase. For example, when nitrous oxide is used as the refrigerant 119b, it is stored at approximately 5.17 MPa (750 psi) to maintain the liquid phase at room temperature. Other suitable refrigerants may be stored at more moderate pressures (e.g., R-410A can be stored at approximately 1.45 MPa (210 psi)). As such, the container 119a can have a corresponding minimum burst pressure (e.g., 10-50 MPa) to withstand the internal pressure of the liquid refrigerant 119b. The container 119a can also hermetically seal the refrigerant 119b therein to prevent or reduce refrigerant leakage (e.g., less than 1 g/year). For example, the refrigerant cartridge 118 can include a polymer seal (e.g., a polymer grommet), a crimping closure, a fused cap (e.g., a metal welded cap with a maximum pierce force of 500 N), and/or other suitable hermetic sealing mechanisms. Such hermetic seals can also reduce the likelihood that moisture and/or other contaminants will enter the refrigerant cartridge 118.

In operation, the refrigerant cartridge 118 with the high purity refrigerant 119b may provide a reliable and efficacious supply of refrigerant for cryotherapeutic neuromodulation. The relatively small cartridge 118 can be moved manually and can be easily set on or proximate a patient during treatment, thereby enhancing the flexibility of the system 100. For example, the cartridge 118 can be inserted into a handle 130 of the system 100. Additionally, the small cartridge 118 reduces the amount of residual refrigerant 119b that remains in the container 119a after cryotherapy. Accordingly, the residual refrigerant 119b can be vented from the container 119a after treatment such that the container 119a can be disposed of without special handling (e.g., as is commonly necessary with larger refrigerant reservoirs). Moreover, the clean refrigerant 119b stored in the container 119a may have a purity and a moisture content that inhibit contaminants from reaching the dew point and freezing during neuromodulation at cryotherapeutic temperatures (e.g., less than −60° C.). This reduces or prevents obstructions in the small supply lumen 106 that is used to access renal arteries. Accordingly, the use of the single-use or refillable refrigerant cartridge 118 may reduce the likelihood of failure of the system 100 during cryotherapeutic renal neuromodulation.

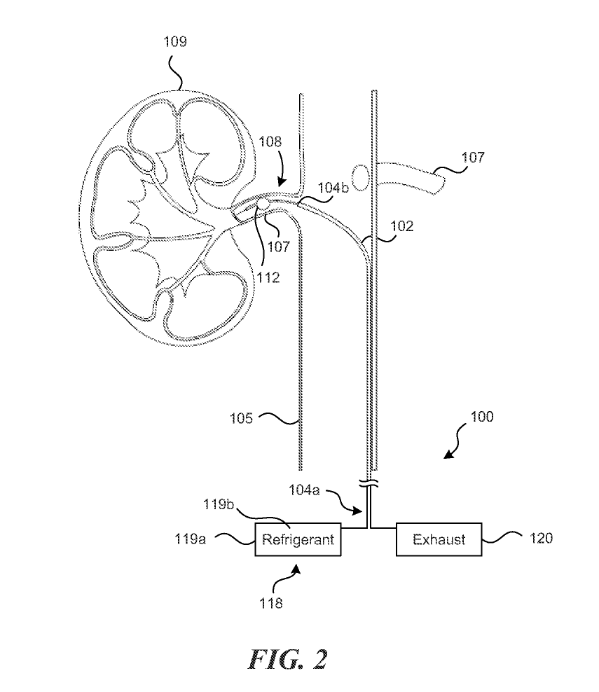

FIG. 2 is a partially schematic view of the system 100 of FIG. 1 performing cryotherapeutic renal nerve modulation in accordance with an embodiment of the present technology. As shown, the distal portion 104b of the shaft 102 can be located intravascularly in a renal artery 107 via the aorta 105 (e.g., via a femoral, brachial, radial, axillary or other artery, not shown). The substantially pure refrigerant 119b from the container 119a can be transported to the distal portion 104b of the shaft 102 via the supply lumen 106 (FIG. 1) and expanded from a liquid phase to a gas phase into the expansion chamber 112 via the orifice 110 to cause therapeutically-effective cryomodulation to neural fibers that innervate the kidney 109. Such neuromodulation can be performed while the renal artery 107 is partially or fully occluded, and can be applied around a full or partial circumference of the renal artery 107 in one or more applications. In various aspects of the technology, the cooling assembly 108 can be retracted into a delivery state (e.g., a low-profile or collapsed configuration) and moved from one renal artery 107 to the opposite renal artery 107, where the cooling assembly 108 can be moved to a deployed state (e.g., an expanded configuration) to apply therapeutically-effective renal neuromodulation.

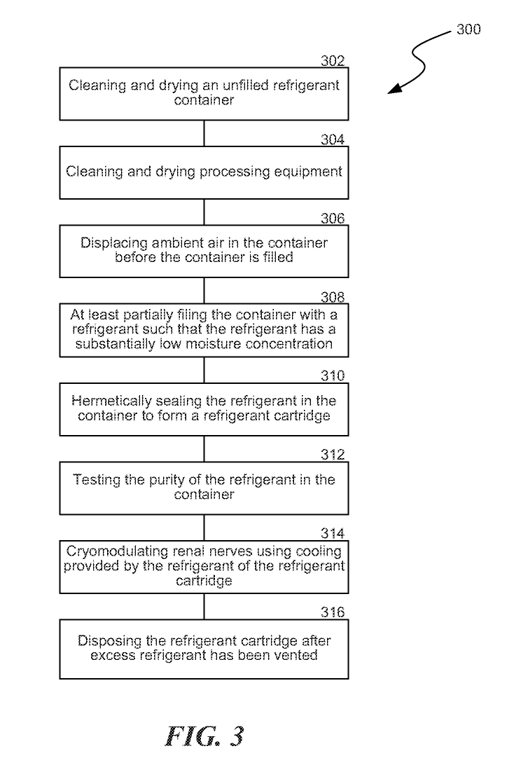

FIG. 3 is a block diagram illustrating a method 300 of preparing a refrigerant cartridge in accordance with an embodiment of the present technology. The refrigerant cartridge can include features generally similar to those of the refrigerant cartridge 118 described above with reference to FIGS. 1 and 2. For example, the refrigerant cartridge can include a small container having a volume of a substantially pure refrigerant sufficient for at least one cryotherapeutic treatment. The method 300 of preparing the refrigerant cartridge can include cleaning and drying an unfilled container (block 302) and associated processing equipment (block 304) to reduce the presence of moisture and/or other contaminants on the container before it is filled with the refrigerant. In various embodiments, cleaning can be performed in a dry environment to reduce the moisture content on and/or in the container. For example, the container may be cleaned using vacuum baking, an ultra dry process gas (e.g., a gas having a moisture concentration of less than 2 ppm), and/or other suitable cleaning processes.

The method 300 can further include displacing the ambient air from the container before it is filled using a vacuum or other suitable displacement mechanism (block 306). The container can then be partially or fully filled with a substantially pure refrigerant (e.g., a refrigerant having a purity of approximately 95% or higher) while maintaining a relatively low moisture concentration (e.g., 10 ppm) in the refrigerant (block 308). For example, in one embodiment the container can be filled with at least 22 grams of nitrous oxide such that the container has a fill density of at most 80% and a moisture concentration of at most 10 ppm. In other embodiments, the container can have more or less refrigerant, a greater or lesser fill density, and/or a greater or lesser moisture concentration (e.g., 40 ppm, 5 ppm, 1 ppm, etc.). The purity of the refrigerant can be maintained before and after filling by using processes and materials for storing containers and/or transporting the refrigerant that reduce the likelihood of contaminants (e.g., moisture) entering the container. In one embodiment, for example, the container can be cleaned, evacuated, and filled within a single manifold device under a vacuum. This reduces the likelihood that the container will reach contaminant equilibrium with the ambient environment, which may occur within fractions of a second after the pre-cleaned container is exposed to the ambient environment. In other embodiments, the unfilled container can be heated (e.g., by vacuum baking) to remove contaminants and then filled within a specified time after heating to reduce or prevent re-equilibration with the ambient environment. For example, the container may be filled within approximately 10 minutes of heating (e.g., within 1-30 seconds of heating) to inhibit layers of contaminants forming on the surface of the container. In further embodiments, the surface of the container can be treated to resist rust, scale, and/or other particulates that may subsequently contaminate the refrigerant stored therein.

The substantially pure refrigerant can then be hermetically sealed within the container at a high pressure in a substantially liquid phase (block 310). For example, nitrous oxide can be stored within the container at a pressure of approximately 5.17 MPa (750 psi), whereas R-410A can be stored at a pressure of approximately 1.45 MPa (210 psi). Other refrigerants can be stored at other suitable pressures that maintain the refrigerant in at least a substantially liquid phase at room temperature. In various embodiments, the amount or type of lubricants and/or other materials used during filling and sealing can be selected to reduce substantial contamination of the refrigerant. For example, the container can be filled without using substantial amounts of lubricating oils that may combine with and contaminate the refrigerant. In other embodiments, the refrigerant cartridge can be crimp-sealed using only a small amount of lubricant.

Optionally, the purity of the refrigerant stored within the container can be tested before use to confirm that the refrigerant stored therein is suitable for cryotherapy (block 312). For example, the particle count of various refrigerants in a batch of refrigerant cartridges can be tested by filtering an effluent wash through the unfilled container and/or filtering effluent refrigerant after the container has been filled. Additionally, dew point testing can be performed to confirm that the moisture concentration of the refrigerant does not induce condensation at cryogenic temperatures used during neuromodulation. In other embodiments, the contamination and/or dew point of the refrigerant in the container can be determined using other suitable methods.

Once the refrigerant is sealed in the container, the refrigerant cartridge can be used as a refrigerant source when cryomodulating renal nerves (block 314). After cryotherapy, residual refrigerant gas can be vented from the container such that it can be disposed of without further special handling (block 316). In certain aspects of the technology, the system can be configured to automatically vent any remaining refrigerant in a safe and efficient manner before disposal. In other embodiments, the refrigerant cartridge can be vented manually and discarded. Accordingly, the method 300 may produce high pressure refrigerant cartridges that are appropriate for use with cryotherapeutic systems for renal neuromodulation, while still maintaining a relatively low manufacture cost that permits the cartridge to be a disposable, single-use device. In various other aspects of the technology, the refrigerant cartridge can be re-treated using the method 300 and refilled for use during subsequent cryotherapy sessions.

### III. RELATED ANATOMY AND PHYSIOLOGY

The sympathetic nervous system (SNS) is a branch of the autonomic nervous system along with the enteric nervous system and parasympathetic nervous system. It is always active at a basal level (called sympathetic tone) and becomes more active during times of stress. Like other parts of the nervous system, the SNS operates through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic) neurons.

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release norepinephrine. Prolonged activation may elicit the release of adrenaline from the adrenal medulla. Once released, norepinephrine binds adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The SNS is responsible for up and down regulation of many homeostatic mechanisms in living organisms. Fibers from the SNS innervate tissues in almost every organ system, providing at least some regulatory function to physiological features as diverse as pupil diameter, gut motility, and urinary output. This response is also known as the sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the SNS and indirectly via catecholamines secreted from the adrenal medulla.

Science typically looks at the SNS as an automatic regulation system, that is, one that operates without the intervention of conscious thought. Some evolutionary theorists suggest that the SNS operated in early organisms to maintain survival as the SNS is responsible for priming the body for action. One example of this priming is in the moments before waking, in which sympathetic outflow spontaneously increases in preparation for action.

A. The Sympathetic Chain

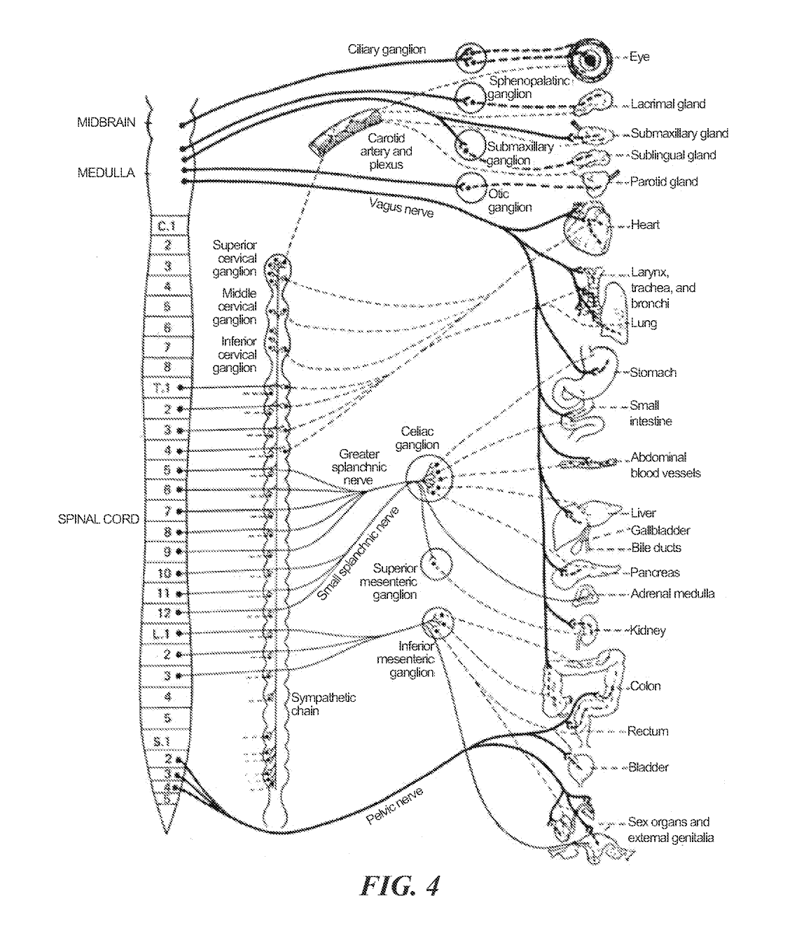

As shown in FIG. 4, the SNS provides a network of nerves that allows the brain to communicate with the body. Sympathetic nerves originate inside the vertebral column, toward the middle of the spinal cord in the intermediolateral cell column (or lateral horn), beginning at the first thoracic segment of the spinal cord and are thought to extend to the second or third lumbar segments. Because its cells begin in the thoracic and lumbar regions of the spinal cord, the SNS is said to have a thoracolumbar outflow. Axons of these nerves leave the spinal cord through the anterior rootlet/root. They pass near the spinal (sensory) ganglion, where they enter the anterior rami of the spinal nerves. However, unlike somatic innervation, they quickly separate out through white rami connectors that connect to either the paravertebral (which lie near the vertebral column) or prevertebral (which lie near the aortic bifurcation) ganglia extending alongside the spinal column.

In order to reach the target organs and glands, the axons travel long distances in the body. Many axon cells relay their messages to second cells through synaptic transmission. For example, the ends of axon cells can link across a space (i.e., a synapse) to dendrites of the second cell. The first cell (the presynaptic cell) can send a neurotransmitter across the synaptic cleft where it activates the second cell (the postsynaptic cell). The message is then carried to the final destination. In the SNS and other components of the PNS, these synapses are made at sites called ganglia, discussed above. The cell that sends its fiber is called a preganglionic cell, while the cell whose fiber leaves the ganglion is called a postganglionic cell. As mentioned previously, the preganglionic cells of the SNS are located between the first thoracic (T1) segment and third lumbar (L3) segments of the spinal cord. Postganglionic cells have their cell bodies in the ganglia and send their axons to target organs or glands. The ganglia include not just the sympathetic trunks but also the cervical ganglia (superior, middle, and inferior), which send sympathetic nerve fibers to the head and thorax organs, and the celiac and mesenteric ganglia, which send sympathetic fibers to the gut.

B. Innervation of the Kidneys

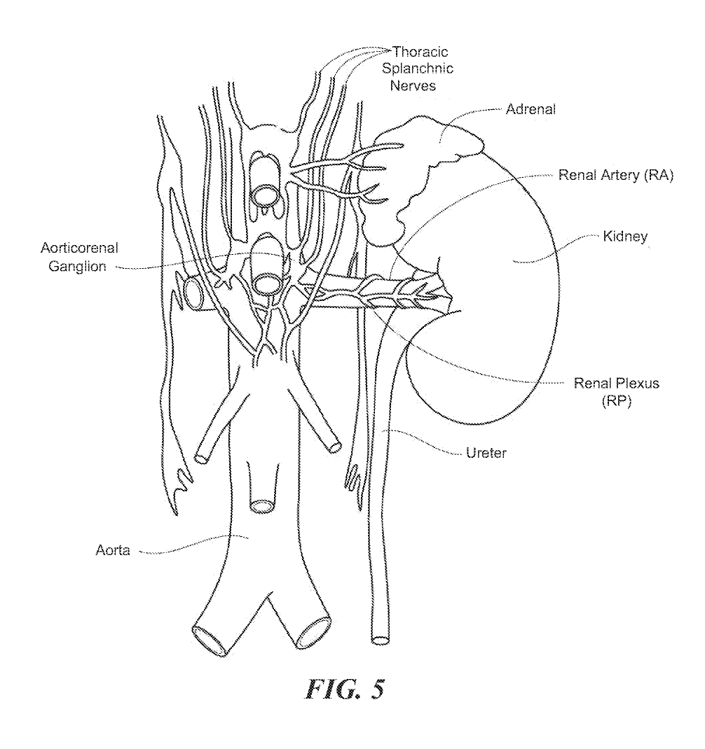

As FIG. 5 shows, the kidney is innervated by the renal plexus, which is intimately associated with the renal artery. The renal plexus is an autonomic plexus that surrounds the renal artery and is embedded within the adventitia of the renal artery. The renal plexus extends along the renal artery until it arrives at the substance of the kidney. Fibers contributing to the renal plexus arise from the celiac ganglion, the superior mesenteric ganglion, the aorticorenal ganglion and the aortic plexus. The renal plexus, also referred to as the renal nerve, is predominantly comprised of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia (they do not synapse) to become the lesser splanchnic nerve, the least splanchnic nerve, the first lumbar splanchnic nerve, and the second lumbar splanchnic nerve, and they travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus and are distributed to the renal vasculature.

C. Renal Sympathetic Neural Activity

Messages travel through the SNS in a bidirectional flow. Efferent messages may trigger changes in different parts of the body simultaneously. For example, the SNS may accelerate heart rate, widen bronchial passages, decrease motility (movement) of the large intestine, constrict blood vessels, increase peristalsis in the esophagus, cause pupil dilation, cause piloerection (i.e., goose bumps), cause perspiration (i.e., sweating), and raise blood pressure. Afferent messages carry signals from various organs and sensory receptors in the body to other organs and, particularly, the brain.

Hypertension, heart failure, and chronic kidney disease are a few of many disease states that result from chronic activation of the SNS, especially the renal sympathetic nervous system. Chronic activation of the SNS is a maladaptive response that drives the progression of these disease states. Pharmaceutical management of the renin-angiotensin-aldosterone system (RAAS) has been a longstanding, but somewhat ineffective, approach for reducing overactivity of the SNS.

As mentioned above, the renal sympathetic nervous system has been identified as a major contributor to the complex pathophysiology of hypertension, states of volume overload (such as heart failure), and progressive renal disease, both experimentally and in humans. Studies employing radiotracer dilution methodology to measure overflow of norepinephrine from the kidneys to plasma revealed increased renal norepinephrine spillover rates in patients with essential hypertension, particularly so in young hypertensive subjects, which in concert with increased norepinephrine spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced SNS overactivity.

Activation of cardiorenal sympathetic nerve activity is even more pronounced in heart failure, as demonstrated by an exaggerated increase of norepinephrine overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration rate, and left ventricular ejection fraction. These findings support the notion that treatment regimens that are designed to reduce renal sympathetic stimulation have the potential to improve survival in patients with heart failure.

Both chronic and end-stage renal disease are characterized by heightened sympathetic nervous activation. In patients with end-stage renal disease, plasma levels of norepinephrine above the median have been demonstrated to be predictive for both all-cause death and death from cardiovascular disease. This is also true for patients suffering from diabetic or contrast nephropathy. There is compelling evidence suggesting that afferent signals originating from the diseased kidneys are major contributors to initiating and sustaining elevated central sympathetic outflow in this patient group. This facilitates the occurrence of the well-known adverse consequences of chronic sympathetic overactivity, such as hypertension, left ventricular hypertrophy, ventricular arrhythmias, sudden cardiac death, insulin resistance, diabetes, and metabolic syndrome.

(i) Renal Sympathetic Efferent Nerve Activity

Sympathetic nerves to the kidneys terminate in the blood vessels, the juxtaglomerular apparatus and the renal tubules. Stimulation of the renal sympathetic nerves causes increased renin release, increased sodium (Na⁺) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation is likely a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), and diuretics (intended to counter the renal sympathetic mediated sodium and water retention). However, the current pharmacologic strategies have significant limitations including limited efficacy, compliance issues, side effects, and others.

(ii) Renal Afferent Nerve Activity

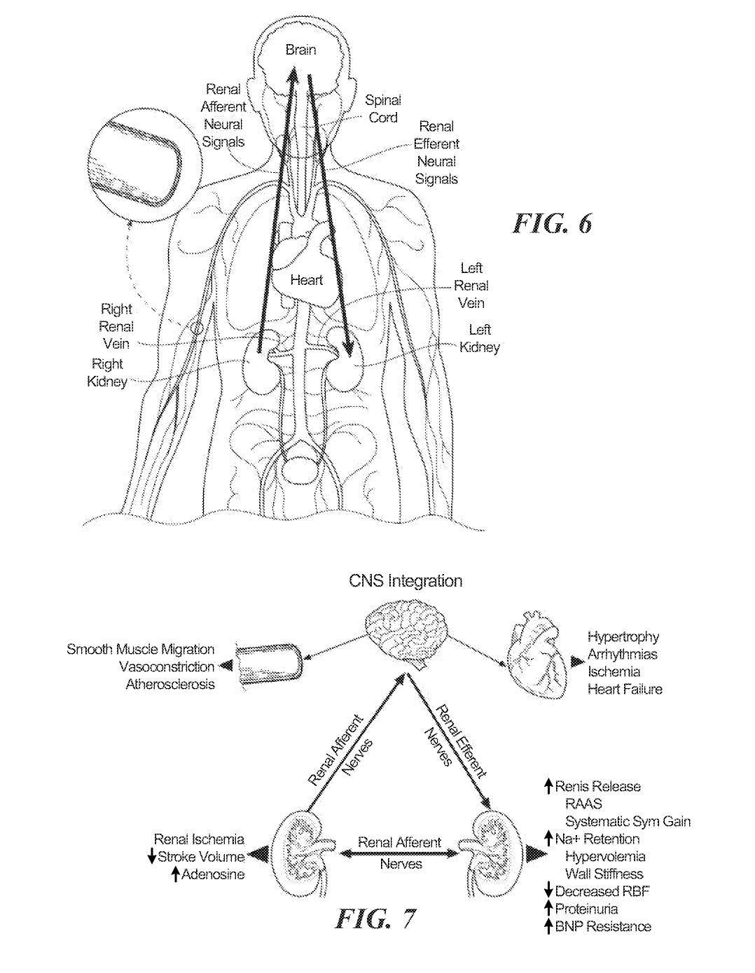

The kidneys communicate with integral structures in the CNS via renal afferent nerves. Several forms of “renal injury” may induce activation of afferent signals. For example, renal ischemia, reduction in stroke volume or renal blood flow, or an abundance of adenosine enzyme may trigger activation of afferent neural communication. As shown in FIGS. 6 and 7, this afferent communication might be from the kidney to the brain or might be from one kidney to the other kidney (via the CNS). These afferent signals are centrally integrated and may result in increased sympathetic outflow. This sympathetic drive is directed toward the kidneys, thereby activating the RAAS and inducing increased renin secretion, sodium retention, volume retention, and vasoconstriction. Central sympathetic overactivity also impacts other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (a) modulation of tissue with efferent sympathetic nerves will reduce inappropriate renin release, salt retention, and reduction of renal blood flow, and (b) modulation of tissue with afferent nerves will reduce the systemic contribution to hypertension and other disease states associated with increased central sympathetic tone through its direct effect on the posterior hypothalamus as well as the contralateral kidney. In addition to the central hypotensive effects of afferent renal denervation, a desirable reduction of central sympathetic outflow to various other sympathetically innervated organs such as the heart and the vasculature is anticipated.

D. Additional Clinical Benefits of Renal Denervation

As provided above, renal denervation is likely to be valuable in the treatment of several clinical conditions characterized by increased overall and particularly renal sympathetic activity such as hypertension, metabolic syndrome, insulin resistance, diabetes, left ventricular hypertrophy, chronic end-stage renal disease, inappropriate fluid retention in heart failure, cardio-renal syndrome, and sudden death. Since the reduction of afferent neural signals contributes to the systemic reduction of sympathetic tone/drive, renal denervation might also be useful in treating other conditions associated with systemic sympathetic hyperactivity. Accordingly, renal denervation may also benefit other organs and bodily structures innervated by sympathetic nerves, including those identified in FIG. 4. For example, as previously discussed, a reduction in central sympathetic drive may reduce the insulin resistance that afflicts people with metabolic syndrome and Type II diabetes. Additionally, patients with osteoporosis are also sympathetically activated and might also benefit from the down regulation of sympathetic drive that accompanies renal denervation.

E. Achieving Intravascular Access to the Renal Artery

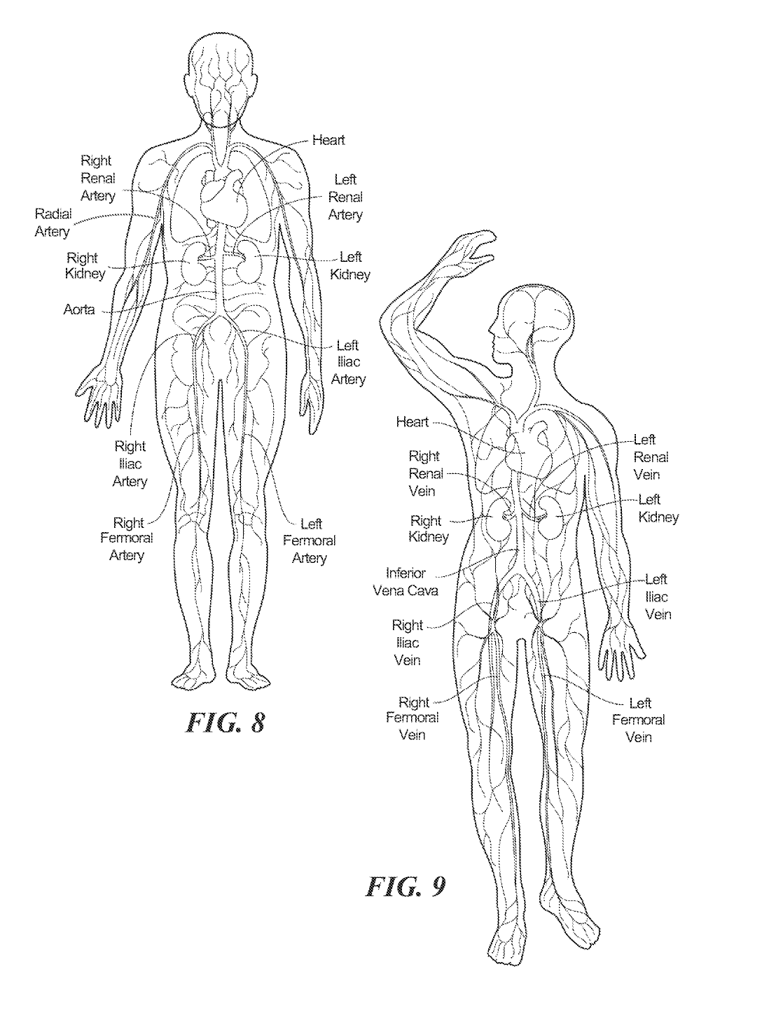

In accordance with the present technology, neuromodulation of a left and/or right renal plexus, which is intimately associated with a left and/or right renal artery, may be achieved through intravascular access. As FIG. 8 shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

As FIG. 9 shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

As will be described in greater detail later, the femoral artery may be accessed and cannulated at the base of the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder region provide other locations for introduction of catheters into the arterial system. For example, catheterization of either the radial, brachial, or axillary artery may be utilized in select cases. Catheters introduced via these access points may be passed through the subclavian artery on the left side (or via the subclavian and brachiocephalic arteries on the right side), through the aortic arch, down the descending aorta and into the renal arteries using standard angiographic technique.

F. Properties and Characteristics of the Renal Vasculature

Since neuromodulation of a left and/or right renal plexus may be achieved in accordance with embodiments of the present technology through intravascular access, properties and characteristics of the renal vasculature may impose constraints upon and/or inform the design of apparatus, systems, and methods for achieving such renal neuromodulation. Some of these properties and characteristics may vary across the patient population and/or within a specific patient across time, as well as in response to disease states, such as hypertension, chronic kidney disease, vascular disease, end-stage renal disease, insulin resistance, diabetes, metabolic syndrome, etc. These properties and characteristics, as explained herein, may have bearing on the efficacy of the procedure and the specific design of the intravascular device. Properties of interest may include, for example, material/mechanical, spatial, fluid dynamic/hemodynamic, and/or thermodynamic properties.

As discussed previously, a catheter may be advanced percutaneously into either the left or right renal artery via a minimally invasive intravascular path. However, minimally invasive renal arterial access may be challenging, for example, because as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Apparatus, systems, and methods for achieving renal neuromodulation via intravascular access can account for these and other aspects of renal arterial anatomy and its variations across the patient population when minimally invasively accessing a renal artery.

In addition to complicating renal arterial access, specifics of the renal anatomy also complicate establishment of stable contact between neuromodulatory apparatus and a luminal surface or wall of a renal artery. When the neuromodulatory apparatus includes a cryotherapeutic device, consistent positioning, appropriate contact force applied by the cryotherapeutic device to the vessel wall, and adhesion between the cryo-applicator and the vessel wall can be important for predictability. However, navigation can be impeded by the tight space within a renal artery, as well as tortuosity of the artery. Furthermore, establishing consistent contact can be complicated by patient movement, respiration, and/or the cardiac cycle because these factors may cause significant movement of the renal artery relative to the aorta, and the cardiac cycle may transiently distend the renal artery (i.e., cause the wall of the artery to pulse).

After accessing a renal artery and facilitating stable contact between a neuromodulatory apparatus and a luminal surface of the artery, nerves in and around the adventitia of the artery can be modulated via the neuromodulatory apparatus. Effectively applying thermal treatment from within a renal artery is non-trivial given the potential clinical complications associated with such treatment. For example, the intima and media of the renal artery are highly vulnerable to thermal injury. As discussed in greater detail below, the intima-media thickness separating the vessel lumen from its adventitia means that target renal nerves may be multiple millimeters distant from the luminal surface of the artery. Sufficient energy can be delivered to or heat removed from the target renal nerves to modulate the target renal nerves without excessively cooling or heating the vessel wall to the extent that the wall is frozen, desiccated, or otherwise potentially affected to an undesirable extent. A potential clinical complication associated with excessive heating is thrombus formation from coagulating blood flowing through the artery. Given that this thrombus may cause a kidney infarct, thereby causing irreversible damage to the kidney, thermal treatment from within the renal artery can be applied carefully. Accordingly, the complex fluid mechanics and thermodynamic conditions present in the renal artery during treatment, particularly those that may impact heat transfer dynamics at the treatment site, may be important in applying energy (e.g., heating thermal energy) and/or removing heat from the tissue (e.g., cooling thermal conditions) from within the renal artery.

The neuromodulatory apparatus can be configured to allow for adjustable positioning and repositioning of an energy delivery element within the renal artery since location of treatment may also impact clinical efficacy. For example, it may be tempting to apply a full circumferential treatment from within the renal artery given that the renal nerves may be spaced circumferentially around a renal artery. In some situations, full-circle lesions likely resulting from a continuous circumferential treatment may be potentially related to renal artery stenosis. Therefore, the formation of more complex lesions along a longitudinal dimension of the renal artery via the cryotherapeutic devices and/or repositioning of the neuromodulatory apparatus to multiple treatment locations may be desirable. It should be noted, however, that a benefit of creating a circumferential ablation may outweigh the potential of renal artery stenosis, or the risk may be mitigated with certain embodiments or in certain patients, and creating a circumferential ablation could be a goal. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging. Manipulation of a device in a renal artery can also consider mechanical injury imposed by the device on the renal artery. Motion of a device in an artery, for example by inserting, manipulating, negotiating bends and so forth, may contribute to dissection, perforation, denuding intima, or disrupting the interior elastic lamina.

Blood flow through a renal artery may be temporarily occluded for a short time with minimal or no complications. However, occlusion for a significant amount of time can be avoided in some cases to prevent injury to the kidney such as ischemia. It can be beneficial to avoid occlusion altogether or, if occlusion is beneficial, to limit the duration of occlusion, for example, to 2-5 minutes.

Based on the above-described challenges of (1) renal artery intervention, (2) consistent and stable placement of the treatment element against the vessel wall, (3) effective application of treatment across the vessel wall, (4) positioning and potentially repositioning the treatment apparatus to allow for multiple treatment locations, and (5) avoiding or limiting duration of blood flow occlusion, various independent and dependent properties of the renal vasculature that may be of interest include, for example, (a) vessel diameter, vessel length, intima-media thickness, coefficient of friction, and tortuosity, (b) distensibility, stiffness, and modulus of elasticity of the vessel wall, (c) peak systolic, end-diastolic blood flow velocity, as well as the mean systolic-diastolic peak blood flow velocity, and mean/max volumetric blood flow rate, (d) specific heat capacity of blood and/or of the vessel wall, thermal conductivity of blood and/or of the vessel wall, and/or thermal connectivity of blood flow past a vessel wall treatment site and/or radiative heat transfer, (e) renal artery motion relative to the aorta induced by respiration, patient movement, and/or blood flow pulsatility, and (f) the takeoff angle of a renal artery relative to the aorta. These properties will be discussed in greater detail with respect to the renal arteries. However, dependent on the apparatus, systems, and methods utilized to achieve renal neuromodulation, such properties of the renal arteries also may guide and/or constrain design characteristics.

As noted above, an apparatus positioned within a renal artery can conform to the geometry of the artery. Renal artery vessel diameter, D_{RA}, typically is in a range of about 2-10 mm (0.079-0.394 inch), with most of the patient population having a D_{RA} of about 4 mm (0.157 inch) to about 8 mm (0.315 inch) and an average of about 6 mm (0.236 inch). Renal artery vessel length, L_{RA}, between its ostium at the aorta/renal artery juncture and its distal branchings, generally is in a range of about 5-70 mm (0.200-2.756 inches), and a significant portion of the patient population is in a range of about 20-50 mm (0.787-1.979 inch). Since the target renal plexus is embedded within the adventitia of the renal artery, the composite intima-media thickness (i.e., the radial outward distance from the artery's luminal surface to the adventitia containing target neural structures) also is notable and generally is in a range of about 0.5-2.5 mm (0.020-0.098 inch), with an average of about 1.5 mm (0.059 inch). Although a certain depth of treatment can be important to reach the target neural fibers, the treatment typically is not too deep (e.g., the treatment can be less than about 5 mm (0.200 inch) from inner wall of the renal artery) so as to avoid non-target tissue and anatomical structures such as the renal vein.

An additional property of the renal artery that may be of interest is the degree of renal motion relative to the aorta induced by respiration and/or blood flow pulsatility. A patient's kidney, which is located at the distal end of the renal artery, may move as much as four inches cranially with respiratory expulsion. This may impart significant motion to the renal artery connecting the aorta and the kidney. Accordingly, the neuromodulatory apparatus can have a unique balance of stiffness and flexibility to maintain contact between a cryo-applicator or another thermal treatment element and the vessel wall during cycles of respiration. Furthermore, the takeoff angle between the renal artery and the aorta may vary significantly between patients, and also may vary dynamically within a patient (e.g., due to kidney motion). The takeoff angle generally may be in a range of about 30-135°.

The foregoing embodiments of cryotherapeutic devices are configured to accurately position the cryo-applicators in and/or near the renal artery and/or renal ostium via a femoral approach, transradial approach, or another suitable vascular approach. In any of the foregoing embodiments described above with reference to FIGS. 1-3, single balloons can be configured to be inflated to diameters of about 3 mm (0.118 inch) to about 8 mm (0.315 inch), and multiple balloons can collectively be configured to be inflated to diameters of about 3 mm (0.118 inch) to about 8 mm (0.315 inch), and in several embodiments 4 mm (0.157 inch) to 8 mm (0.315 inch). Additionally, in any of the embodiments described herein with reference to FIGS. 1-9, the balloons can individually and/or collectively have a length of about 3 mm (0.118 inch) to about 15 mm (0.591 inch), and in several embodiments about 5 mm (0.200 inch). For example, several specific embodiments of the devices described with reference to in FIGS. 1-3 can have a 5 mm (0.200 inch) long balloon that is configured to be inflated to a diameter of 4 mm (0.157 inch) to 8 mm (0.315 inch). The shaft of the devices described above with reference to any of the embodiments described with reference to FIGS. 1-3 can be sized to fit within a 6 Fr sheath, such as a 4 Fr shaft size.

### IV. EXAMPLES

1. A cryotherapeutic system, comprising:
a shaft including a proximal portion and a distal portion, wherein the shaft is configured to locate the distal portion intravascularly at a treatment site;
a supply lumen along at least a portion of the shaft, wherein the supply lumen is configured to receive a refrigerant in a substantially liquid phase;
a refrigerant cartridge at the proximal portion of the shaft in fluid communication with the supply lumen, wherein the refrigerant cartridge is configured to supply the refrigerant to the supply lumen, and wherein the refrigerant has a moisture concentration while in the refrigerant cartridge of at most 10 ppm; and
a cooling assembly at the distal portion of the shaft, the cooling assembly having an expansion chamber in fluid communication with the supply lumen.

2. The cryotherapeutic system of example 1 wherein the refrigerant in the refrigerant cartridge has a contaminant concentration and a normal boiling point, and wherein a dew point of the contaminant concentration is less than the normal boiling point.

3. The cryotherapeutic system of example 1 or example 2 wherein:
the cooling assembly is configured to deliver therapeutically-effective cooling at a temperature of less than −80° C.; and
the refrigerant cartridge includes a refrigerant having a moisture concentration of at most 6 ppm.

4. The cryotherapeutic system of any of examples 1-3 wherein the refrigerant cartridge includes a container having an internal volume between approximately 30 cc and approximately 100 cc, and wherein the refrigerant is in at least a substantially liquid phase in the container and has a normal boiling point of at most −60° C. and a purity of at least 95%.

5. The cryotherapeutic system of any of examples 1-3 wherein the refrigerant is in at least a substantially liquid phase in the refrigerant cartridge and has a purity of at least 98%.

6. The cryotherapeutic system of any of examples 1-3 and 5 wherein the refrigerant cartridge comprises a container and the refrigerant is within the container, and wherein the container includes a volume of the refrigerant sufficient to cryomodulate neural fibers that innervate a kidney around a circumference of a renal artery.

7. The cryotherapeutic system of any of the preceding examples, further comprising a handle at the proximal portion of the shaft, wherein the refrigerant cartridge fits substantially within the handle.

8. The cryotherapeutic system of any of the preceding examples wherein the refrigerant is at least one of nitrous oxide, carbon dioxide, and hydrofluorocarbon.

9. The cryotherapeutic system of any of the preceding examples wherein:
the shaft has an outer diameter of at most 6 Fr; and
the supply lumen includes a capillary tube at the distal portion of the shaft, the capillary tube having a distal end that defines an orifice having a diameter of approximately 0.102 mm (0.004 inch) to approximately 0.203 mm (0.008 inch).

10. A method of making a refrigerant cartridge for a cryotherapeutic treatment, the method comprising:
cleaning a refrigerant container;
at least partially filling the refrigerant container with a refrigerant having a contaminant concentration in the refrigerant container and a normal boiling point, wherein a dew point of the contaminant concentration is less than the normal boiling point of the refrigerant; and
sealing the refrigerant in the refrigerant container to define the refrigerant cartridge.

11. The method of example 10 wherein the refrigerant container has an internal volume between approximately 30 cc and approximately 100 cc.

12. The method of example 11 wherein:
at least partially filling the refrigerant container with the refrigerant comprises at least partially filling the refrigerant container with a substantially liquid phase of at least one of nitrous oxide, carbon dioxide, and hydrofluorocarbon, the refrigerant having a moisture concentration of at most 10 ppm;
sealing the refrigerant in the refrigerant container comprises hermetically sealing the refrigerant in the refrigerant container such that the refrigerant cartridge has a leak rate of at most 1 g/year; and
the method further comprises—
   cleaning processing equipment associated with filling and sealing the refrigerant container, wherein the cleaning is performed in a substantially dry environment, and
   displacing ambient air within the refrigerant container before at least partially filling the refrigerant container.

13. The method of example 10, further comprising cleaning processing equipment used to at least partially fill the refrigerant container and to seal the refrigerant in the refrigerant container, wherein the cleaning is performed in a substantially dry environment.

14. The method of any of examples 10-13, further comprising testing the purity of the refrigerant after at least partially filling the refrigerant container, wherein the refrigerant has a purity of at least 95%.

15. The method of any of examples 10, 11, 13 and 14 wherein at least partially filling the refrigerant container comprises at least partially filling the refrigerant container with a liquid phase refrigerant including at least one of nitrous oxide, carbon dioxide, and hydrofluorocarbon.

16. The method of any of examples 10-15 wherein:
cleaning the refrigerant container comprises heating the refrigerant container; and
at least partially filling the refrigerant container comprises at least partially filling the refrigerant container with the refrigerant within 10 minutes of heating the refrigerant container.

17. The method of example 16 wherein at least partially filling the refrigerant container comprises at least partially filling the refrigerant container with the refrigerant within 1 minute of heating the refrigerant container.

18. The method of any of examples 10-17 wherein cleaning and at least partially filling the refrigerant container comprises cleaning and at least partially filling the refrigerant container within a single device in a vacuum.

19. The method of any of examples 10-18, further comprising:
coupling the refrigerant cartridge to a proximal portion of a supply lumen of a cryotherapeutic device, wherein the supply lumen is in fluid communication with a cooling assembly at a distal portion of the supply lumen; and
cryomodulating renal nerves with the cooling assembly of the cryotherapeutic device using the refrigerant.

20. The method of example 19 wherein cryomodulating renal nerves with the cooling assembly comprises intravascularly locating the cooling assembly of the cryotherapeutic device in a delivery state at a renal vessel or renal ostium, the cooling assembly having a size of at most 6 Fr in the delivery state.

21. The method of example 19 or example 20, further comprising venting excess refrigerant from the refrigerant container after cryomodulation.

22. A method of treating a patient, the method comprising:
intravascularly positioning a cooling assembly proximate a renal vessel or renal ostium;
supplying a refrigerant in at least a substantially liquid phase from a refrigerant cartridge to a proximal portion of a supply lumen, the supply lumen being in fluid communication with the cooling assembly at a distal portion of the supply lumen, wherein the refrigerant has a purity of at least 95% in the refrigerant cartridge;
expanding the refrigerant at the cooling assembly; and
cryomodulating at least a portion of neural fibers that innervate a kidney proximate the cooling assembly.

23. The method of example 22 wherein supplying the refrigerant comprises supplying at least one of nitrous oxide, carbon dioxide, and hydrofluorocarbon.

24. The method of example 22 wherein supplying the refrigerant comprises supplying a refrigerant having a contaminant concentration in the refrigerant cartridge and a normal boiling point, and wherein a dew point of the contaminant concentration is less than the normal boiling point of the refrigerant.

25. The method of any of examples 22-24 wherein:
supplying the refrigerant comprises supplying a refrigerant having a moisture concentration of at most 10 ppm; and
cryomodulating at least a portion of neural fibers that innervate a kidney comprises generating temperatures of −60° C. or lower in the cooling assembly.

26. The method of any of examples 22-24 wherein:
supplying the refrigerant comprises supplying a refrigerant having a moisture concentration of at most 5 ppm; and
cryomodulating at least a portion of neural fibers that innervate a kidney comprises generating temperatures of −80° C. or lower in the cooling assembly.

27. The method of any of examples 22-26, further comprising:
venting excess refrigerant from a container of the refrigerant cartridge after cryomodulation; and
disposing the container after a single use.

### V. CONCLUSION

The above detailed descriptions of embodiments of the present technology are for purposes of illustration only and are not intended to be exhaustive or to limit the present technology to the precise form(s) disclosed above. Various equivalent modifications are possible within the scope of the present technology, as those skilled in the relevant art will recognize. For example, while stages may be presented in a given order, alternative embodiments may perform stages in a different order. The various embodiments described herein and elements thereof may also be combined to provide further embodiments. In some cases, well-known structures and functions have not been shown or described in detail to avoid unnecessarily obscuring the description of embodiments of the present technology.

Where the context permits, singular or plural terms may also include the plural or singular terms, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout the disclosure to mean including at least the recited feature(s) such that any greater number of the same feature(s) and/or additional types of other features are not precluded. It will also be appreciated that various modifications may be made to the described embodiments without deviating from the present technology. Further, while advantages associated with certain embodiments of the present technology have been described in the context of those embodiments, other embodiments may also exhibit such advantages, and not all embodiments need necessarily exhibit such advantages to fall within the scope of the present technology. Accordingly, the disclosure and associated technology can encompass other embodiments not expressly shown or described herein.

I/We claim:

1. A cryotherapeutic system, comprising:
a shaft including a proximal portion and a distal portion, wherein the shaft is configured to locate the distal portion intravascularly at a treatment site;
a supply lumen along at least a portion of the shaft, wherein the supply lumen is configured to receive a refrigerant in a substantially liquid phase;
a refrigerant cartridge at the proximal portion of the shaft in fluid communication with the supply lumen, wherein the refrigerant cartridge is configured to supply the refrigerant to the supply lumen, and wherein the refrigerant has a moisture concentration while in the refrigerant cartridge of at most 10 ppm; and
a cooling assembly at the distal portion of the shaft, the cooling assembly having an expansion chamber in fluid communication with the supply lumen.

2. The cryotherapeutic system of claim 1 wherein the refrigerant in the refrigerant cartridge has a contaminant concentration and a normal boiling point, and wherein a dew point of the contaminant concentration is less than the normal boiling point.

3. The cryotherapeutic system of claim 1 or claim 2 wherein:
the cooling assembly is configured to deliver therapeutically-effective cooling at a temperature of less than −80° C.; and
the refrigerant cartridge includes a refrigerant having a moisture concentration of at most 6 ppm.

4. The cryotherapeutic system of any of claims 1-3 wherein the refrigerant cartridge includes a container having an internal volume between approximately 30 cc and approximately 100 cc, and wherein the refrigerant is in at least a substantially liquid phase in the container and has a normal boiling point of at most −60° C. and a purity of at least 95%.

5. The cryotherapeutic system of any of claims 1-3 wherein the refrigerant is in at least a substantially liquid phase in the refrigerant cartridge and has a purity of at least 98%.

6. The cryotherapeutic system of any of claims 1-3 and 5 wherein the refrigerant cartridge comprises a container and the refrigerant is within the container, and wherein the container includes a volume of the refrigerant sufficient to cryomodulate neural fibers that innervate a kidney around a circumference of a renal artery.

7. The cryotherapeutic system of any of the preceding claims, further comprising a handle at the proximal portion of the shaft, wherein the refrigerant cartridge fits substantially within the handle.

8. The cryotherapeutic system of any of the preceding claims wherein the refrigerant is at least one of nitrous oxide, carbon dioxide, and hydrofluorocarbon.

9. The cryotherapeutic system of any of the preceding claims wherein:
the shaft has an outer diameter of at most 6 Fr; and
the supply lumen includes a capillary tube at the distal portion of the shaft, the capillary tube having a distal end that defines an orifice having a diameter of approximately 0.102 mm (0.004 inch) to approximately 0.203 mm (0.008 inch).

10. A method of making a refrigerant cartridge for a cryotherapeutic treatment, the method comprising:
cleaning a refrigerant container;
at least partially filling the refrigerant container with a refrigerant having a contaminant concentration in the refrigerant container and a normal boiling point, wherein a dew point of the contaminant concentration is less than the normal boiling point of the refrigerant; and
sealing the refrigerant in the refrigerant container to define the refrigerant cartridge.

11. The method of claim 10 wherein the refrigerant container has an internal volume between approximately 30 cc and approximately 100 cc.

12. The method of claim 11 wherein:
at least partially filling the refrigerant container with the refrigerant comprises at least partially filling the refrigerant container with a substantially liquid phase of at least one of nitrous oxide, carbon dioxide, and hydrofluorocarbon, the refrigerant having a moisture concentration of at most 10 ppm;
sealing the refrigerant in the refrigerant container comprises hermetically sealing the refrigerant in the refrigerant container such that the refrigerant cartridge has a leak rate of at most 1 g/year; and
the method further comprises—
cleaning processing equipment associated with filling and sealing the refrigerant container, wherein the cleaning is performed in a substantially dry environment, and
displacing ambient air within the refrigerant container before at least partially filling the refrigerant container.

13. The method of claim 10, further comprising cleaning processing equipment used to at least partially fill the refrigerant container and to seal the refrigerant in the refrigerant container, wherein the cleaning is performed in a substantially dry environment.

14. The method of any of claims 10-13, further comprising testing the purity of the refrigerant after at least partially filling the refrigerant container, wherein the refrigerant has a purity of at least 95%.

15. The method of any of claims 10, 11, 13 and 14 wherein at least partially filling the refrigerant container comprises at least partially filling the refrigerant container with a liquid phase refrigerant including at least one of nitrous oxide, carbon dioxide, and hydrofluorocarbon.

16. The method of any of claims 10-15 wherein:
cleaning the refrigerant container comprises heating the refrigerant container; and
at least partially filling the refrigerant container comprises at least partially filling the refrigerant container with the refrigerant within 10 minutes of heating the refrigerant container.

17. The method of claim 16 wherein at least partially filling the refrigerant container comprises at least partially filling the refrigerant container with the refrigerant within 1 minute of heating the refrigerant container.

18. The method of any of claims 10-17 wherein cleaning and at least partially filling the refrigerant container comprises cleaning and at least partially filling the refrigerant container within a single device in a vacuum.

19. The method of any of claims 10-18, further comprising:
coupling the refrigerant cartridge to a proximal portion of a supply lumen of a cryotherapeutic device, wherein the supply lumen is in fluid communication with a cooling assembly at a distal portion of the supply lumen; and
cryomodulating renal nerves with the cooling assembly of the cryotherapeutic device using the refrigerant.

20. The method of claim 19 wherein cryomodulating renal nerves with the cooling assembly comprises intravascularly locating the cooling assembly of the cryotherapeutic device in a delivery state at a renal vessel or renal ostium, the cooling assembly having a size of at most 6 Fr in the delivery state.

21. The method of claim 19 or claim 20, further comprising venting excess refrigerant from the refrigerant container after cryomodulation.

22. A method of treating a patient, the method comprising:
intravascularly positioning a cooling assembly proximate a renal vessel or renal ostium;
supplying a refrigerant in at least a substantially liquid phase from a refrigerant cartridge to a proximal portion of a supply lumen, the supply lumen being in fluid communication with the cooling assembly at a distal portion of the supply lumen, wherein the refrigerant has a purity of at least 95% in the refrigerant cartridge;
expanding the refrigerant at the cooling assembly; and
cryomodulating at least a portion of neural fibers that innervate a kidney proximate the cooling assembly.

23. The method of claim 22 wherein supplying the refrigerant comprises supplying at least one of nitrous oxide, carbon dioxide, and hydrofluorocarbon.

24. The method of claim 22 wherein supplying the refrigerant comprises supplying a refrigerant having a contaminant concentration in the refrigerant cartridge and a normal boiling point, and wherein a dew point of the contaminant concentration is less than the normal boiling point of the refrigerant.

25. The method of any of claims 22-24 wherein:
supplying the refrigerant comprises supplying a refrigerant having a moisture concentration of at most 10 ppm; and
cryomodulating at least a portion of neural fibers that innervate a kidney comprises generating temperatures of −60° C. or lower in the cooling assembly.

26. The method of any of claims 22-24 wherein:
supplying the refrigerant comprises supplying a refrigerant having a moisture concentration of at most 5 ppm; and
cryomodulating at least a portion of neural fibers that innervate a kidney comprises generating temperatures of −80° C. or lower in the cooling assembly.

27. The method of any of claims 22-26, further comprising:
venting excess refrigerant from a container of the refrigerant cartridge after cryomodulation; and
disposing the container after a single use.
